(19) United States
(12) Patent Application Publication
(10) Pub. No.: US 2005/0042603 A1
(43) Pub. Date: Feb. 24, 2005

(54) Compositions and methods for peptide-assisted transfection

(75) Inventor: Jiwu Wang, San Diego, CA (US)

Correspondence Address: GRAY CARY WARE & FREIDENRICH LLP, 4365 EXECUTIVE DRIVE, SUITE 1100, SAN DIEGO, CA, 92121-2133, US

(73) Assignee: Allele Biotechnology & Pharmaceuticals, Inc., San Diego, CA (US)

(21) Appl. No.: 10/881,781

(22) Filed: Jun. 29, 2004

Related U.S. Application Data
(60) Provisional application No. 60/484,394, filed on Jul. 1, 2003.

(51) Int. Cl. C12Q 1/70; C07K 14/16; C07H 21/02; C12N 15/867; C12N 7/00

(52) U.S. Cl. 435/5; 435/69.3; 435/235.1; 435/456; 435/325; 530/350; 536/23.72

(57) ABSTRACT

Compositions that increase the efficiency of nucleic acid transfection of cells are provided, including peptide transfection reagents and fusion proteins containing the peptides. Also provided are methods of using the peptide transfection reagents and fusion proteins to transfect cells.

This application claims the benefit of priority under 35 U.S.C. § 119 of U.S. Ser. No. 60/484,394, filed Jul. 1, 2003, the entire content of which is incorporated herein by reference.

### BACKGROUND OF THE INVENTION

1. Field of the Invention

The invention relates generally to peptides that have a desirable non-specific dsDNA binding ability and are useful for facilitating transfection of eukaryotic cells. Also disclosed are various compositions and methods of transfecting eukaryotic cells utilizing such peptides. The invention also relates to compositions and methods of combining peptides with salt and known transfection reagents for transfection.

2. Background Information

Delivery of genetic material into cells is important in many areas of research, including, for example, studies of gene function, genetic pathway analysis, examination of cell morphology in relation to genetic alteration, and cell-based library screening. Delivery of genetic material into cells is also a critical step of gene therapy, which can provide benefit to an organism. Transfection is the standard method by which cells can be induced to take-up nucleic acids and produce polypeptides useful as production units for manufacturing or as scaffolds for tissue engineering (for review, see [Godbey, 2001 #150], the full citation for all references cited herein are listed can be found following the Examples, below). Introduction of the cDNA of genes that play roles in lineage maintenance or commitment can facilitate the use of stem cells for cell-based therapy [Reya, 2003 #151].

Most high efficiency gene deliveries, especially those in clinical studies, rely on viral vectors as a carrier, for example, lentiviral and adenoviral vectors. Factors influencing viral vector usage include lengthy procedures of virus preparations, genome packaging limits, toxicity and safety, and target cell tropism. In many in vitro and ex vivo cases, non-viral gene delivery systems is selected because of its simplicity of use. Genetic material can be delivered by transfection with various techniques, including, for example, calcium precipitation, complexation with cationic lipid, DEAE-dextran, dendrimer polymers, polybrene or other cationic reagents. Delivery can also be mediated by electroporation or a mechanical method such as microinjection or biolistic methods, which can be particle based.

Liposomes and cationic polymers currently make up the two major classes of chemical gene delivery methods. Liposome-mediated transfection provides advantages such as relatively high efficiency in a wide variety of cell types, ability for delivery of DNA of all sizes ranging from oligonucleotides to yeast artificial chromosomes [Lamb, 1995 #152], delivery of RNA [Malone, 1989 #153], and delivery of proteins [Debs, 1990 #156]. DNA transfected into cells by liposomes can be integrated into chromosomes for long-term experiments. There are an increasing number of studies that use liposomes for delivery of nucleic acid to animals and humans [Felgner, 1995 #157][Liu, 1995 #155; Liu, 1997 #154]. However, a major problem with liposome-mediated transfection is that intracellular accumulation of secreted proteoglycans compete with DNA to form complexes with liposome and thereby reduce transfection efficiency [Belting, 1999 #21].

One of the most successful polycations used as transfection reagent is poly(ethylenimine) (PEI). Compared to common liposomes, PEI seems to provide better protection of DNA against nuclease digestion [Ferrari, 1999 #158], while demonstrating equal or better transfection capability in vitro and in vivo. The ability of PEI to compact DNA is believed to be responsible for keeping large cargo DNA molecules (e.g., yeast artificial chromosomes of 2.3 Mb) intact and delivering them into eukaryotic cells [Marschall, 1999 #159]. Unfortunately, a disadvantage of using PEI, as well as several other transfection reagents, is the high level of toxicity to cells exposed to either free or DNA-complexed PEI [Godbey, 2001 #29].

Due to the disadvantages associated with existing transfection system, many other molecules are being tested for gene delivery. Most of these experimental reagents and methods utilize a form of cationic molecule in forming complexes, at physiological pH, with negatively charged DNA molecules, and bringing the DNA into close contact with the negatively charged cell surface. Examples of such reagents include chitosan [Richardson, 1999 #160], and β-cyclodextrin [Gonzalez, 1999 #161]. Various degrees of success have been reported in comparison with those using CaPO4, LIPOFECTAMINE transfection reagent, PEI, and other “classic” reagents.

Regardless of the method chosen for transfecting a particular type of cells, the following obstacles must be overcome for an effective expression of introduced DNA: 1) close contact of DNA-containing complex with cell surface, 2) passage through cell membrane, 3) dismantling of the DNA-encapsulating complexes in the cytoplasm in most cases, and 4) nuclear import of the DNA. The mechanism of cell entry remains unclear for most established transfection methods, although endocytosis is likely a main route for DNA complexes to enter cells. The fate of different DNA-containing complexes, once inside cytoplasm, depends on the type of reagents used to precipitate or surround the DNA molecules. The mechanism for the degradation or dismantling of the complexes also is not fully understood. A tight DNA-encompassing complex may be favorable in surviving the process of contacting and penetrating cell surface, but may not be efficient in releasing DNA for nuclear entrance and gene expression. In addition, reagents that bind DNA often can bind serum components as well, resulting in less transfection in the presence of serum in cell medium or in vivo. An example is the use of cationic lipid oleoyl-ornithate (OLON) in combination with dioleoylphosphatidylethanolamine (DOPE), which gives a higher transfection efficiency than other liposomes, presumably because of thermodynamically more favorable separation of the cargo DNA molecules after cellular entry [Tang, 1999 #162]. As another example, phosphorothioate oligonucleotides delivered by PEI displayed higher antisense activity than similarly delivered phosphodiester oligonucleotides at least in part due to more favorable thermodynamics of releasing carried DNA [Dheur, 1999 #163; Dheur, 2000 #164].

Nuclear entry is another barrier for transgenic expression. By most transfection methods, the best chance for cytoplasmically located DNA to enter the nucleus is during the reorganization of the nuclear membrane during cell division. In non-mitotic cells, nuclear envelop crossing by plasmid DNA is a very rare event [Escriou, 2001 #133]. Accordingly, transfection efficiency with polycation-based or lipid-based transfection systems could be 30-fold to 500-fold higher when transfection is performed during S or G2 phase as compared with cells in G1 phase [Brunner, 2000 #131]. This factor can create a dilemma because many commercially available transfection reagents require the cells be near confluent at the time of transfection, leaving little room for significant cell division to occur following transfection. In addition, it is constantly observed that cells are temporarily arrested, due to the cytotoxicity of most lipid-based or polycation-based reagents, and show less cell division in a given time compared to untreated controls. The arrested cells often recover after growing in reagent-free medium for a day or two. Recent advances in improving nuclear transfer of transfected DNA include the addition of nuclear localization signal (NLS), for instance, a signal peptide derived from SV40, to the DNA/liposome complex [Aronsohn, 1998 #165].

Peptides also can be included in a DNA carrier complex to enhance cell binding through receptor recognition, thereby enhancing transfection. Certain viral proteins or their fragments increase the percentage of transfected cells when included in cationic lipid-based transfection complex (see, for example, Wickham et al. [Wickham, 1995 #166], Yoshimura et al. [Yoshimura, 1993 #167], Kamata et al. [Kamata, 1994 #168], and Remy et al. [Remy, 1995 #169]). Hawley-Nelson et al. (U.S. Pat. Nos. 5,736,392; 6,051,429; and 6,376,248; and U.S. Pat. Appl. Publ. No. 2003/0069173) described a related system, which was stated to be different than the above cited examples because it can significantly improve the efficiency of transfection when peptide is bound to nucleic acid prior to adding the transfection reagent. Hawley-Nelson et al. also state that peptides covalently coupled to the transfection agent, e.g., directly or indirectly linked to a lipid or to a dendrimer, function to improve transfection. It is not clear, however, how the suggested cell surface-binding peptides such as VSVG and RGD can function similarly either pre-bound to DNA or linked to lipid, and how this system differs methodologically from previous protocols. It appears that the effect of the peptides was solely due to the effect of polycation peptides binding to DNA, as the addition of a polycationic peptide without NLS improved transfection even more than that of a cationic NLS. However, it is not clear how these DNA-bound positively charged peptides can also function as NLS or other functional motif during complex transport.

There have been more recent successes in using peptides to direct DNA-containing complexes to the surface of selected cells. Scott et al. synthesized a peptide containing an integrin-binding tripeptide (RGD) and a DNA-binding polylysine for enhancement of liposome-mediated gene delivery [Scott, 2001 #171]. Increased transfection was seen with integrin-expressing cells, and the effect of the RGD peptide on cell contact appeared specific as control RGE or competitive antibodies against integrin reduced gene transfer. By a biotin-streptavidin mediated conjugation scheme, Lee et al. linked PEGylated epidermal growth factor (EGF) to PEI-DNA complex and achieved EGF receptor-mediated endocytosis [Lee, 2002 #172].

There have also been examples of using nuclear location signals in similar manner. Gopal et al. (U.S. Pat. No. 5,670,347) suggested a peptide composed of an NLS, a flexible hinge, and a basic DNA-binding motif; Gerhard et al (DE-OS 195 41 679) described nuclear localization signal- (NLS-) polylysine conjugate for gene transfer; and Szoka (PCT 1993) bound an NLS to DNA via an intercalating chemical agent; Toth et al. used a lipophilic palmitoyl-peptide derived from SV40 T antigen NLS in combination with PEI [Toth, 2002 #49]. In all these cases, DNA-binding is intended to rely on either positively charged peptides or chemical moieties. In either case, it is plausible to expect that cationic NLS binds DNA as well and may not function to their full potential as signal peptide. DNA-binding solely depending on positive charge cannot survive competition by other negatively charged molecules during the process of transfection. In addition, the use of mutagenic intercalators described by Hawley-Nelson et al. limits the application of the methods.

For improvement over these methods, Siebenkotten et al. (U.S. Pat. No. 6,521,456) described a system in which the main modifications was to use a specific DNA binding domain such as a lac repressor or a PNA molecule as the DNA binding module, and a neutralized NLS as the targeting module. A drawback of this method is that for sequence-specific DNA-binding domains, a specific site (or sites) must be engineered into the plasmid, thus limiting a broad use of the reagent. For PNA, a specific PNA-peptide conjugate has to be made for each type of plasmid. Also, there can be no more than a few sequence-specific binding events, by definition, between each DNA and the transfection reagent molecules. Therefore, it is difficult to compact large DNA or to improve transfection efficiency by increase the ratio of peptide/DNA. Even though Siebenkotten et al. disproved the earlier reports of using proteins for transfection, e.g., HMG-1 by Kaneda (U.S. Pat. No. 5,631,237) and thymus histones by Fritz et al. [Fritz, 1996 #170], partly for the reason of high cost and labor of producing proteins compared to peptides, the lac repressor they used is a protein.

More recently, Behr et al. (U.S. Pat. Appl. Publ. No. 2003/0100113) described the use of a nuclear localization signal (NLS) covalently linked to an oligonucleotide, and noted that the NLS conjugate can be covalently linked to one or both termini of a linear DNA molecule, associated with a plasmid DNA molecule by forming a triple helix, or inserted in a plasmid DNA molecule by strand invasion. The transfection vector is useful for gene therapy applications. This system is based on a peptide-oligonucleotide hybrid molecule, which requires specific site on the delivered DNA to match with the oligonucleotide sequence on the hybrid, and chemical conjugation of oligo to peptide. As such, the system is not a very convenient and, therefore, is not likely to be widely used. As a result of these limitations, there is great need to identify materials and methods for gene delivery that can result in high efficiency, low toxicity, low cost, and convenience in broad applications.

### SUMMARY OF THE INVENTION

The present invention provides peptide transfection reagents, compositions that include such peptide transfection reagents, kits containing the transfection reagents and/or compositions, and methods of using the peptide transfection reagents and/or compositions for transfecting a eukaryotic cell with high efficiency. Accordingly, the present invention relates to a peptide transfection reagent having the amino acid sequence QRNPNKKWS (SEQ ID NO:1), which is a peptide fragment of a Nun polypeptide (“Nuc”). In one embodiment, the peptide transfection reagent is a component of a fusion protein, which, in addition to the peptide of SEQ ID NO:1, contains one or more heterologous polypeptides operatively linked thereto In another embodiment, the peptide transfection and one or more heterologous polypeptides are associated via a non-covalent interaction that is stable under physiological conditions, including conditions suitable for performing a transfection reaction.

In one aspect, the heterologous peptide is a cellular localization domain, for example, a nuclear localization signal (e.g., PKKIKTED; SEQ ID NO:4), which facilitates transport of the fusion protein, and any nucleic acid molecule complexed thereto, into the nucleus of a eukaryotic cell. In other aspects, the cellular localization signal comprises an HIV TAT peptide, for example, a TAT peptide having an amino acid sequence including YGRKKRRQRRR (SEQ ID NO:2), which facilitates translocation of the heterologous polypeptide, and any nucleic acid molecule complexed thereto, across a eukaryotic cell membrane and into a cell, or is an HIV gp41 peptide GALFGGFLGAAGSTMGA; SEQ ID NO:5). In still other aspects, the heterologous peptide can comprise a functional sequence such as an endoprotease recognition site (e.g., a cathepsin D recognition sequence; GGFLGF; SEQ ID NO:6), whereby, when a complex comprising a nucleic acid molecule and a peptide comprising the endoprotease recognition sequence is localized in a region of a target cell containing the endoprotease (e.g., a endosome, or cytosol), proteolytic cleavage occurs and some or all of the peptide is removed from the nucleic acid molecule. In another example, the functional heterologous peptide can be an endosomolytic peptide such as the influenza virus fusogenic peptide, INF7 (GLFEAIEGFIENGWEGMIDGWYG; SEQ ID NO:7). In another embodiment, the peptide-nucleic acid complex can include an endosomolytic agent such as chloroquine operatively associated with the complex.

The peptide transfection reagent and one or more heterologous polypeptide (e.g., an HIV TAT peptide (SEQ ID NO:2) or an HIV gp41 peptide (SEQ ID NO:5, or a nuclear localization signal (SEQ ID NO:4), alone or in combination with an HIV TAT peptide or an HIV gp41 peptide) can be operatively linked by directly linking the peptides together, for example, by forming a peptide (or other bond) between the C-terminus of one peptide and the N-terminus of the second (or more) peptide(s), or by expressing the fusion protein from a recombinant nucleic acid molecule encoding the peptide components, in frame, or the peptide transfection reagent and heterologous polypeptide(s) can be operatively linked via a spacer, which can be any molecule useful for linking two or more peptides to each other, for example, an amino acid or peptide linker. A fusion protein of the invention is exemplified by SEQ ID NO:1 operatively linked to SEQ ID NO:2 via a single glycine linker (see SEQ ID NO:3).

A composition of the invention can further include a nucleic acid molecule, which, upon contact with the peptide transfection reagent, forms or is capable of forming a complex. The nucleic acid molecule can be a single stranded or a double stranded nucleic acid molecule, and can be DNA or RNA or a DNA/RNA hybrid. In addition, a composition of the invention can include divalent cations, for example, divalent calcium ions.

The present invention also provide a kit, which contains at least a peptide transfection reagent of the invention, and can further contain reagents useful for performing and/or monitoring a transfection reaction and/or instructions for using the peptide transfection reagent for transfecting a cell. As such, the kit can further contain one or a plurality of heterologous polypeptides, for example, a cellular localization domain or a plurality of different cellular localization domains. Preferably, the heterologous polypeptides are in a form that facilitates an association or operative linkage with the peptide transfection reagent, for example, by having sequences that facilitate an association that is stable under physiological conditions or that facilitate the formation of a covalent linkage to the peptide transfection reagent such that the heterologous polypeptide and peptide transfection reagent each maintains its desired function. If desired, a kit of the invention can further contain one or more reagents for operatively linking the relevant peptides, either directly or via a linker moiety. Where the kit contains a plurality of such heterologous polypeptides, an advantage is provided in that a user of the kit can select an heterologous polypeptide as desired, i.e., depending on the particular needs of the user.

The kit also can contain one or more reagents useful for performing a transfection reaction, including, for example, buffers, transfectable nucleic acid molecules useful a standard (controls) for monitoring transfection efficiency, and the like. In one aspect, the kit contains divalent calcium ions, either in a solution or in a form that can be placed into solution.

The present invention further relates to a method of transfecting a cell. In one embodiment, the method is performed, for example, by contacting the cell, generally a eukaryotic cell (e.g., a mammalian cell such as a human cell) with a peptide transfection reagent as disclosed herein (e.g., SEQ ID NO:1) and a nucleic acid molecule under conditions sufficient from cell transfection. Such conditions can include, for example, an appropriate concentration of divalent calcium ions. In another embodiment, the methods is performed, for example, by contacting the cell with a fusion protein, which includes a peptide transfection reagent operatively linked to a heterologous polypeptide (e.g., a fusion protein as set forth in SEQ ID NO:3), and a nucleic acid molecule under conditions sufficient for cell transfection. Such conditions can include, for example, an appropriate concentration of divalent calcium ions. In still another embodiment, the method is adapted to a high throughput format, whereby, due to the high transfection efficiency obtained using the disclosed compositions, a plurality of cells, which can be the same or different, can be transfected in parallel with one or more polynucleotides (e.g., polynucleotides encoding small interfering RNA molecules), which can be the same or different.

### DETAILED DESCRIPTION OF THE INVENTION

Methods and compositions are provided for delivering genetic material into cells by the function of a short peptide, Nuc, which can bind to double stranded (ds) DNA and dsRNA in a manner that is not substantially due to an electrostatic interaction. As disclosed herein, linkage of the Nuc peptide to a transmembrane domain peptide (e.g., HIV TAT) enhances transfection mediated by other transfection reagents. As further disclosed herein, the Nuc peptide can form complexes with DNA molecules and, therefore, mediate transfection, alone. Unexpectedly, calcium ion stabilized the complex of DNA and Nuc-containing peptide. In combination with calcium ion, but not necessarily calcium phosphate precipitate, the Nuc peptide resulted in transfection of DNA with efficiency higher than, or at least comparable to, commercial transfection reagents under practically the best conditions. The use of the Nuc-containing peptide did not result in any observable cytotoxicity, required a very low amount of DNA and peptide, and was of significantly lower cost than existing lipid-based and polycation-based transfection reagents.

The present invention relates to a protein motif (Nuc), which is derived from Nun, a natural DNA and RNA binding protein of phage HK022 (see GenBank Acc. No. P18683; see, also, GenBank Acc. No. X16093, each of which is incorporated herein by references). Nuc can function independently as a peptide that binds to dsDNA non-sequence-specifically with affinities desirable for carrying nucleic acids into cells and allowing the genetic material to be expressed in the nucleus. The invention also relates to peptides further derived from the core sequence, QRNPNKKWS (SEQ ID NO:1), of the Nuc peptide. For example, peptides that have more than one repeat of the core sequence in a linear or branched form, peptides with limited residue substitutions, peptides that link the core sequence(s) to other functional motifs such as to a protein transduction domain (PTD), other fusagenic peptides, a nuclear localization signal (NLS), receptor or surface protein binding domain, and charged peptides such polyarginine or polylysine. Derived peptides also can include those that have certain modified amino acids such as amino acids that are partially deprotected after chemical synthesis, or unnatural (non-naturally occurring) L-amino acids. Further, one or a few, e.g., 2, 3, 4, 5, or 6, amino acids can be linked to one or both ends of the Nuc peptide to provide a desired characteristic (e.g., a cysteine residue can be linked via a peptide bond to a terminus of the Nuc peptide (SEQ ID NO:1) to facilitate cross-linking of the Nuc peptide with another Nuc peptide or other peptide of interest). The invention also relates to modifications that result in additional intercalating of the peptide to dsDNA or dsRNA, for example, a modification including one or more added aromatic amino acids.

Prior to the present invention, one main family of DNA-binding peptides was reported in the literature in regard to DNA transfection. These DNA binding peptides were based on having positively charged amino acids often spaced in viral protein transduction domain (PTD). Such examples include peptide JTS1 or GALA [Rittner, 2002 #56] and related KALA [Wyman, 1997 #57], CL-22 [Haines, 2001 #17]. Because the most commonly used viral PTDs contain numerous positively charged amino acids, and are overall basic, there were a few reports of using them directly as a transfection vehicle. For instance, HIV TAT [Sandgren, 2002 #4] and HIV VPR [Coeytaux, 2003 #174] has been reported to carry DNA into cells, although many of these studies only showed indirect measurement of the transfection events. To the other extreme, simple repeats of cationic amino acids such as polyarginine have been tried for similar purposes [Kim, 2003 #1]. These peptides have two fundamental shortcomings: first, they are pH-dependent as protonation status influences the mainly electrostatic interactions between the charged amino acids and DNA; and second, these peptides do not distinguish dsDNA from ssDNA, RNA, certain serum proteins, polyanionic heparan sulfate proteoglycans or other charged molecules in general [Sandgren, 2002 #4], making them somewhat nonspecific carriers that can deliver unintended cargo molecules into cells or animals.

It was therefore desirable to find other types of short peptide domains that specifically recognize dsDNA (and dsRNA) in a sequence-nonspecific manner, and particularly with a medium range of affinity. Since dsDNA is the form of transgene in most cases, it can be beneficial to have binding biased toward dsDNA, thus minimizing, if desired, transfection of ssDNA and most RNA. Recent development in the RNA interference (RNAi) field has illuminated the need to transfect dsRNA into cells. It is therefore also desirable to have a peptide that also can bind dsRNA by non-electrostatic means. Binding in a sequence-independent manner can avoid the need for including a specific site as in the method of Siebenkotten et al (U.S. Pat. No. 6,521,456), and can allow multiple binding such that a stable complex can be formed and the DNA compacted for transmembrane movement and protected from nuclease attacks. A moderate binding affinity provides the greatest likelihood of creating a tight enough complex between transfection agent and DNA for transfection, and of releasing the DNA once inside the intended location, generally the nucleus, for gene expression.

In an effort to identify a short peptide that binds dsDNA by mainly a non-static interaction, the C-terminal 9 amino acid region of phage HK022 Nun protein was found to meet the specified requirements [Watnick, 2000 #44]. Extensively studied, this region interacts directly with dsDNA. Nun does not arrest polymerase on ssDNA template, indicating that its DNA binding motif only binds dsDNA. Point mutations in the C-terminal region have illustrated its role for dsDNA binding and pinpointed the penultimate residue, tryptophan (W 108), as the most important residue for binding. The fact that W108 can be functionally replaced only by other aromatic amino acids (e.g., tyrosine) indicates that binding is due, at least in part, to intercalation of W108 into dsDNA. As such, this domain also should bind dsRNA. Importantly, binding of nucleic acid molecules by the peptide does not seem to have any detectable sequence preference.

The present invention also relates to other peptide domains that share the desirable characteristics as described above and exemplified by the Nuc peptide. Other non-sequence-specific DNA binding proteins or domains that can be used as a transfection reagent as disclosed herein include, for example, the region of amino acid residues 22 to 44 of mouse intermediate filament (IF) protein vimentin [Shoeman, 1999 #68], wherein DNA binding can be mediated by intercalation into dsDNA. Other such domains include those from HU proteins [Grove, 2001 #69], HMG1/2 [Saito, 1999 #70], DNA topoisomerase I, and the like, which exhibit the above described properties desired of a transfection reagent of the invention.

The present invention also provides composition that includes a DNA-binding peptide as disclosed herein alone, or in combination with or conjugated to other peptide domains. The selected or designed DNA binding domain can be linked, for example, to a second functional domain that can facilitate movement of a peptide-DNA complex across a biological membrane. As such, the peptide transfection reagent (e.g., Nuc as set forth in SEQ ID NO:1) can be operatively linked and/or operatively associated with a second (or more) heterologous polypeptide. The term “operatively linked” or “operatively associated” is used herein with respect to two or more molecules that share a covalent or non-covalent interaction, wherein each molecule maintains all or most of a function that the molecule exhibits alone. As such, a nucleotide sequence encoding a first peptide, e.g., Nuc, can be operatively linked to a nucleotide sequence encoding a heterologous peptide, e.g., a cellular localization domain, wherein, upon expression, the nucleotide sequences are in frame and can be expressed either as a linked fusion protein or as two independent peptides that can associate via a non-covalent interaction. Similarly, a first peptide and a second peptide or other molecule can be combined, for example, in a reaction mixture, such that a covalent bond or non-covalent interaction can be formed linking or associating, respectively, the two peptides, wherein each component in the complex maintains a desired function characteristic of the component in a non-complexed form. It should be recognized that a nucleic acid molecule complexed with a peptide transfection reagent of the invention also can be considered to operatively associated because, in such a complex, the peptide transfection reagent maintains its function of facilitating uptake of the nucleic acid into a cell and the nucleic acid molecule maintains its function of encoding an RNA and, if appropriate, polypeptide. As such, operatively associated molecules as disclosed herein generally are stable when exposed to physiological conditions as occur, for example, in a transfection medium, a cell culture medium, or in a cell or cellular compartment.

A heterologous polypeptide operatively linked or operatively associated with a peptide transfection reagent of the invention can be any polypeptide that is not linked or associated with the peptide in nature. In one embodiment, the heterologous polypeptide is a cell localization domain, which can facilitate transport of the operatively linked peptide transfection reagent and any nucleic acid molecule complexed therewith, to a particular compartment of a cell. Cell localization domains are well known in the art and include, for example, a plasma membrane localization domain, a nuclear localization signal, a mitochondrial membrane localization signal, an endoplasmic reticulum localization signal, or the like, or a PTD such as the cationic human immunodeficiency virus (HIV) TAT PTD or the non-charged HIV gp41 PTD, each of which can facilitate translocation of a peptide linked thereto into a cell (see Schwarze et al., Science 285:1569-1572, 1999; Derossi et al., J. Biol. Chem. 271:18188, 1996; Hancock et al., EMBO J. 10:4033-4039, 1991; Buss et al., Mol. Cell. Biol. 8:3960-3963, 1988; U.S. Pat. No. 5,776,689; Morris et al., Nucl. Acids Res. 25:2703-2736, 1997; Morris et al Nucl. Acids Res. 27:3510-3517, 1999; each of which is incorporated herein by reference).

A nuclear localization signal (NLS) facilitates translocation of a nucleic acid molecule, for example, a polydeoxyribonucleic acid molecule that is transcribed by RNA polymerase III and encodes an siRNA, into the nucleus of a eukaryotic cell. Traditionally, it was believed that DNA in the cytoplasm moves by diffusion, and that about 1 in 3,000 molecules enter the nucleus through the nuclear pores (Zanta et al., Proc. Natl. Acad. Sci., USA 96: 91-96, 1999); cell division provides the best opportunity for transfected DNA to be enveloped inside the nucleus. Recently, real-time multiple particle tracking revealed that PEI-DNA nanocomplexes can move towards the nucleus by motor protein-driven transport (Suh et al., Proc. Natl. Acad. Sci., USA 100: 3878-3882, 2003), suggesting that, in addition to random movement, DNA molecules also are actively transported to the proximity of the nuclear membrane by a network involving microtubules. In the presence of microtubule depolymerizing agent nocodazole or vinblastine, the nucleus-bound movement of DNA was significantly hindered (Coonrod et al., Gene Ther. 4: 1313-1321, 1997;Suh et al., supra, 2003). In addition, an NLS can increase transfection efficiency as an additive to lipofection or cationic polymer complexes (Branden et al., Nat. Biotechnol. 17:784-787, 1999). In another case, multiple copies of SV40 NLS were used directly as transfection agent (Ritter et al., J. Mol. Med. 81:708-717, 2003, apparently through static interaction between the positively charged peptide and DNA. Whether NLS peptides already bound to DNA also can associate with the nuclear pore complex for nuclear entry is unclear.

An NLS conveniently can be included as a component of the disclosed peptide transfection agents However, most of the well-defined NLS peptides are cationic (review by Nakielny and Dreyfuss, Cell 99:677-690, 1999), and, therefore, may irreversibly bind DNA and adversely affect transfection. In such a case, non-cationic NLS peptides such as two such sequences present in human DNA topoisomerase I (Mo et al., J. Biol. Chem. 275: 41107-41107, 2000) can be used in the present compositions. One of the topoisomerase I NLS peptides is an acidic amino acids-rich, 29 residue peptide, and the other has 2 neutral, 2 acidic, and only 3 basic amino acids in a 7 residue sequence. Both NLS peptides displayed strong NLS activity in natural and reporter proteins. As disclosed herein, the 7 residue topoisomerase I NLS peptide (KKIKTED; SEQ ID NO:8) does not irreversibly condense DNA and, in view of its short size, can be conveniently synthesized for inclusion in a composition of the invention.

In another embodiment, a heterologous peptide operatively linked to a peptide transfection reagent facilitates disruption of the nucleic acid-peptide complex, thereby releasing the nucleic acid from all or a part of the peptide transfection reagent. Such peptides are exemplified herein by peptides that comprise an endoprotease recognition site and by peptides having endosomolytic activity. There are several families of endoproteases that are abundant in the cytoplasmic compartments with peptide cutting abilities. Such proteases can be used to advantage in order to release the nucleic acid from bound peptides inside the cell. A similar strategy was utilized for plasmid transfection with cationic peptides, although the specific effects of the protease site was not illustrated (Haines et al., Gene Ther. 8:99-110, 2001).

An endoprotease recognition site useful in a composition of the invention is selected based on the intracellular compartment(s) in which the complexes pass through or localize (e.g., endosomes, endoplasmic reticulum, and Golgi body), or on the presence of proteases expression in target cells (e.g., caspases in cells that are subject to apoptosis). For example, the cleavage site GGFLGF (SEQ ID NO:6) of the endosomal endoprotease, cathepsin D, can be placed between a PTD (e.g., SEQ ID NO:2 or SEQ ID NO:5) or other peptide component of the complex, and the Nuc (SEQ ID NO:1) peptide, wherein, upon uptake of the complex via an endosome-mediated pathway, the endosomal cathepsin D cleaves and releases a portion of the peptide from the complex. The efficacy of such a method can be confirmed, for example, by labeling the nucleic acid molecule and the amino end of the peptide with different fluorescent dyes to observe their localization inside cells in a time course. Peptides with or without the endoprotease cleavage site can be compared using a fluorescent dye assay, wherein detecting separation of the peptide and DNA after a certain time lapse only when the peptide contains the cleavage site confirms that the peptide is cleaved.

Whether transfection of particular cell types involves endocytosis can be examined by comparing transfection at lower temperature, and/or in the presence of endocytosis inhibiting agents such as cytochalasin B and balfilmycin A. Upon confirming that the transfection process involves endocytosis, an endosomolytic agent can be included in the nucleic acid-peptide complex. Such endosomolytic peptides are exemplified by INF7 (GLFEAIEGFIENGWEGMIDGWYG; SEQ ID NO:7; Ritter et al. 2003, J. Mol. Med. 81, 708-17; Plank et al. 2002, J. Biol. Chem. 277, 2437-2443, each of which is incorporated herein by reference), and by mellitin (Ogris et al., J. Biol. Chem. 276: 47550-47555, 2001, which is incorporated herein by reference). INF7, for example, is a fusogenic peptide from influenza virus. INF7 can be operatively linked to another peptide in the complex (e.g., via a disulfide bond or by expression as a fusion protein), or can be operatively associated with the complex (e.g., via a hydrophobic interaction). In another embodiment, the endosomolytic agent is a non-peptide agents such as chloroquine, which can be operatively linked to or associated with the complex.

A detectable label, which facilitates identification of a composition of the invention or of a sample or cell containing the composition, also can be operatively linked to a peptide transfection reagent, or peptide linked thereto. The detectable label can be a peptide, polypeptide, or chemical or small organic or inorganic molecule that can be conveniently detected. For example, a detectable label can be a molecule such as a biotin, which can be detected using avidin or streptavidin; a fluorescent compound (e.g., Cy3, Cy5, Fam, fluorescein, or rhodamine); a radionuclide (e.g., sulfur-35, technicium-99, phosphorus-32, or tritium); a paramagnetic spin label (e.g., carbon-13); a bioluminescent such as luciferin; an enzyme such as alkaline phosphatase; or a chemiluminescent compound. Methods of operatively linking a detectable label or other moiety to a nucleotide sequence are well known in the art (see, for example, Hermanson, “Bioconjugate Techniques”(Academic Press 1996), which is incorporated herein by reference). In addition to providing a means, for example, to detect a cell containing the peptide transfection reagent, a detectable label or other moiety also can be used to isolate such a cell. For example, where the fusion protein includes an operatively linked fluorescent compound, cells containing the peptide transfection reagent and, therefore, that contain a nucleic acid molecule complexed with the reagent, can be isolated from cells that do not contain the peptide/nucleic acid complex by a methods such as fluorescent activated cell sorting (FACS). Similarly, where the detectable label is a peptide tag such as a myc epitope, FLAG epitope, or the like, an antibody or other binding partner specific for the tag, which itself can be labeled, can be used to isolate or otherwise identify a cell containing the peptide/nucleic acid complex.

In one embodiment, the nine residue Nun C-terminal peptide (Nuc; SEQ ID NO:1) was fused to a 11 residue TAT peptide (SEQ ID NO:2), wherein the peptides were separated by a single glycine residue spacer. As disclosed herein, the fusion protein (designated TAN; SEQ ID NO:3) bound to double stranded DNA as a multimer, with an apparent dissociation constant of about 1×10⁻⁵ M to 1×10⁻⁴ M. When binding to plasmid DNA, the peptide appears to compact the DNA molecule into a more mobile form, as it migrates faster on gel. This result contrasts with that observed for previously used peptides having DNA-compacting ability, which caused the complexed DNA to stay in loading wells. A smeared, but distinguishable group of complexes were observed when TAN bound to short linear dsDNA generated by PCR. These DNA-binding characteristics may have a role in the ability of Nuc-containing peptides to increase transfection efficiency.

When TAN was mixed with plasmid DNA in a simple water-only reaction, then applied to cultured cells, a few cells expressed the reporter gene carried on the plasmid, indicating that the peptide facilitates transfection and, therefore, acts as a transfection reagent. Accordingly, the invention provides a transfection reagent, compositions that include the transfection reagent, and transfection procedure utilizing the transfection reagent.

Remarkably, divalent calcium ions (Ca⁺⁺) supplied in the transfection reaction using CaCl₂ resulted in transfection of nearly all of the cells in the culture. When green fluorescent protein (GFP) was used as reporter gene, the transfected cells appeared to express the reporter gene to a higher level as compared to cells transfected using other methods. Another advantage of the reagents and methods of the invention is that a time course study revealed that the transfection complex to form quickly (after about only one minute), thus providing a very fast procedure. Additionally, it took less time for the transfected genes to be expressed using the peptides and methods of the invention as compared to liposome or other commercial transfection systems. The treated cells did not show arrest of growth or cell death that typically is observed after treatment with several liposome or PEI transfection reagents, indicating that the transfection reagents of the invention exhibit low toxicity.

Agreeing with the result of using calcium ion for Nuc-mediated transfection, calcium ion stabilized the peptide-DNA complex in a band-shift assay. The dramatic enhancement of transfection by calcium ion can be due, at least in part, to its effect on the complex formation between multiple peptide and DNA. Of note, no phosphate was added for this effect, thus demonstrating that a calcium phosphate precipitate does not have a significant role in the observed results, though the possibility that calcium ion somehow helps opening up the cell membrane cannot be ruled out.

The invention also relates to further usage of the peptides of the invention in combination with other transfection reagent. A several fold increase of transfection efficiency was consistently observed when the peptide transfection reagent was bound to DNA first, then mixed with liposome-based transfection reagent.

The invention further relates to compositions including a DNA-binding peptide domain transfection reagent operatively linked to (or operatively associated with) one or more (e.g., 2, 3, 4, etc.) other peptide domains and/or other molecules for transfection into particular cell types or in animals or humans. Such domains include, but are not limited, to PTD, NLS, endoprotease, and endosomolytic sequences or molecules, as discussed above, as well as natural or artificial domains that can destabilize or pass through a biological membrane. PTDs have been derived from HIV TAT protein (TAT) [Becker-Hapak, 2001 #39], the homeodomain of Drosophila transcription factor Antennapedia, the HSV protein VP22, basic fibroblast growth factor, and HIV gp41, etc. Other membrane-active peptide include melittin and the like. A few peptides also can posses similar properties by design, including, for example, MPG, Pep-1, and oligomers of L-arginine and D-arginine, lysine, and histidine. TAT has been used to deliver large fusion proteins into various cells and adult animals, and also crosses the blood-brain barrier effectively [Schwarze, 1999 #10].

Also provided are kits, which include at least a transfection reagent comprising the Nuc peptide for transfecting eukaryotic cells. A kit of the invention also can contain one or more additional reagents useful for practicing or monitoring a transfection reaction including, for example, a calcium ion source (e.g., CaCl₂), a standard nucleic acid molecule, or complex thereof, useful for monitoring transfection efficiency, buffers, or other such materials typically used in a transfection reaction. In addition, the kit can contain one or more other peptides, as desired, for example, a TAT peptide, a nuclear localization signal, and the like, which can be separate component of the kit, thus providing a means to select and complex the Nuc peptide to the other peptide, or can be in the form of a fusion peptide with the Nuc peptide.

The methods and compositions of the invention can be useful for transfecting cells in vitro, including cells adapted to culture (e.g., cell lines or panels of cells that have adapted to culture) or cells ex vivo (i.e., cells that have been removed from a subject such as a human subject for the purpose of manipulating (transfecting) the cells in culture, expanding (if desired) the manipulated cells, and re-administering the cells back into the same or a different, generally a haplotype-matched, subject). In addition, the methods and compositions of the invention are useful for introducing a nucleic acid molecule into cells of a subject in vivo, thus providing methods for performing animal studies, including for example, developing transgenic animals, which can express a desired gene product or provide a desired animal model of a disease, particularly a human disease, as well as providing methods of human gene therapy. It is known that most of the widely available cationic lipids, including, for example, LIPOFECTAMINE transfection reagent and DC-cholesterol, have a very poor ability to enhance DNA expression above the baseline level with naked DNA in animals [Felgner, 1995 #157; Ferrari, 1999 #158]. From studies of TDP and NLS functions, it is clear that peptides possess and extremely potent ability to bring macromolecules into cells and organs of animals. As such, peptides as disclosed herein, in combination with a peptide transfection reagent of the invention and, if desired, other reagents, can be used for enhancing gene transfer by non-viral means.

The methods of the present invention can be conveniently adapted to a high throughput format, thereby allowing for two or more transfection reactions to be performed in parallel. Accordingly, the present invention also provides methods of performing a plurality (i.e., 2 or more) transfection reactions in a high throughput format, including, for example, on a solid support, wherein individual and discrete reactions can be performed. The solid support can be any substrate typically used for performing a high throughput assay (e.g., a silicon wafer, a glass slide, or a bead), and the samples can, but need not, be arranged in an array, which can be an addressable array. Microarray technology has been applied to many areas of biomedical analysis, including for monitoring gene expression, genotyping single nucleotide polymorphisms (SNP), and sequencing.

The principle of microarrays of cells was recently established (Ziauddin and Sabatini, Nature 411:107-110, 2001), thus allowing expression of a defined cDNA in a cluster of cells grown on a slide upon which hundreds of cDNAs in expression vectors were spotted. The success of this methods depends on the cDNA molecules being “printed” on the slides in such a way that they can be moved away from the solid support (i.e., into a cell), and the cDNA molecules being in form suitable for internalization by cells. In the original methods, cDNA was mixed with a gelatin solution before spotting onto the slide. After drying, the cDNA spots were exposed to transfection reagents, then the slides were placed in a culture dish and overlaid with adherent mammalian cells in medium. Because of the reversed order-of-addition of DNA molecules and cells, this process is referred to as “reverse transfection”.

Cell arrays have been successfully applied to RNAi analysis based on the reverse transfection principles, and using the gelatin method as originally described, or using sucrose and MATRIGEL matrix for embedding siRNAs complexed with lipofection agents before spotting (Kumar et al., Genome Res. 13:2333-2340, 2003; Mousses et at., Genome Res. 13:2341-2347, 2003, each of which is incorporated herein by reference). Unfortunately, these embedding methods have two major shortcomings. First, DNA spotted in gelatin-like solutions is not highly confined and, therefore, are not conveniently adaptable for generating high density microarrays and, generally, are limited to the use of a few hundreds siRNA spots per slide, which is far below the upper limit set by the attainable cell density (Ziauddin and Sabatini, supra, 2001). For comparison, the hybridization DNA chips can be printed at 1 million features per chip (e.g., a GeneChip™ microarray; Affymetrix). This limitation prevents the use of the current RNAi chips for genome-wide or random RNAi library screening. The second limitation of the reverse transfection method relates to the transfection efficiency. A typical transfection reagent seldom gives 100% efficiency under normal tissue culture conditions, and is much lower when the DNA is embedded in a semisolid carrier protein layer. Unfortunately, a high transfection efficiency generally is required to obtain meaningful down-regulation by RNAi, as compared to that required to observe positive gene expression by cDNA.

As disclosed herein, linear DNA cassettes encoding siRNAs can be used to generate high density arrays. Instead of semisolid embedding, the linear cassette molecules is immobilized by one end onto a slide, similar to any chip containing hybridizing oligonucleotides. For the immobilized DNA to be internalized by cell, it is printed via a transmembrane domain (TMD) peptide with an intramembrane cleaving protease site (I-CliPs). I-CliPs are a rapidly expending family of proteases and peptidases that unexpectedly hydrolyze substrate proteins or peptides within the hydrophobic environment of membrane lipid bilayers Wolfe and Selkoe, Science 296:2156-2157, 2002). The presenilin 1 and presenilin 2 I-CliPs are selected for exemplifying the present methods because they are nearly ubiquitously expressed, and can cut peptides within the cell membrane (other I-CliPs cleave within ER or Golgi membranes) so as to release the cytosolic portion of the substrate into the cytoplasm or, in some cases, into the nucleus Lee et al., J. Neurosci. 16: 7513-7525, 1996). In the exemplified method, the TMD peptide is synthesized with a biotin group at one end, linked to the DNA at the other. The biotinylated molecule can be spotted to streptavidin-coated chips at a high density commensurate to the optimum growth density of the cells. The TMD can be cleaved by I-CliPs once the DNA is uplifted into a cell.

A TMD sequence can be prepared using standard procedures, and an N-terminal biotin can be added using biotin-N-hydroxysuccinimide ester. A linear cassette of about 125 bp encoding an siRNA can be generated by oligonucleotide synthesis, which each strand synthesized to contain a 5′ amino group and conjugated to a cysteine residue on either the TMD or the PTD-containing peptide. Crosslinking can be achieved using Sulfo-SMCC reagent. Peptide-conjugated sense and antisense strands are hybridized prior to spotting onto streptavidin-coated slides. Alternatively, the linear nucleic acid cassettes can be conjugated only to the TMD peptide (and not the PTD peptide), then, after spotting, the chip is soaked in solutions containing the nucleic acid-peptide complex. The cells then can be overlaid. Such methods for preparing the microarrays, including, for example, spotting the nucleic acid-peptide complex onto the substrate and contacting the arrays with cells, can be performed manually, or can be partially or fully automated, as can further steps of examining the transfected cells for expression of the introduced nucleic acid molecule.

The following examples are provided to illustrate aspects of the present invention.

### EXAMPLES

There are many potential benefits to using peptide as transfection reagent, including, for example, they are relatively easy to manufacture, store and use; have low toxicity; have low cost; are flexible to use alone, or in combination with other reagents; and are easy to modify and reformulate. Such peptide transfection reagents are exemplified herein by a fusion protein, which is composed of at least two functional components, including a DNA binding/compacting module and a biological membrane affinity/passing module. The effectiveness of the disclosed compositions is demonstrated using the C-terminal domain of the Nun protein (“Nuc”) as the DNA-binding domain, and the HIV TAT as the membrane transduction domain. Although TAT was used because it is a well-studied targeting peptide, other peptides having similar membrane translocating activity similarly can be used. For example, TAT carries a strong positive charge and, therefore, can bind to negatively charged molecules surrounding cells. As such, signal peptides that are not cationic also can be used as a component of a fusion protein including Nuc, such that non-specific binding is reduced. One such example is the NLS of amino acids 117-146 of human DNA topoisomerase I and its counterparts in mouse, hamster, chicken, frog, and other species (Mo et al., J. Biol. Chem. 52:41107-13. 2000). This domain is negatively charged and, therefore, will not bind to many serum proteins and polyglycans. It can be further modified to be neutral to further avoid non-specific binding.

Example 1 demonstrates that a fusion peptide between Nuc and TAT is capable of entering cells. Example 2 illustrates that the fusion peptide, TAN, can bring reporter DNA plasmid into cells as a transfection reagent. Example 3 further shows that the transfection with TAN can be greatly enhanced with the addition of low amount of calcium. Example 4 further shows that TAN can facilitate transfection in lipid- or cationic polymer-based transfection systems. Example 5 provides evidence that the TAN transfection system can be applied to many different cell types, some of which are known to be difficult-to-transfect. Example 6 is the result of a direct binding assay between TAN and dsDNA plasmid. Example 7 shows that TAN can also complex with short linear dsDNA that functions in RNAi.

### Example 1

### Transduction Abilities of Peptide Tan

TAN and TAT having the following sequences were synthesized by Fmoc chemistry (SynPep Corp.; Dublin Calif.):

| | | |
|---|---|---|
| TAT: | YGRKKRRQRRR; | (SEQ ID NO:2) |
| and | | |
| TAN: | YGRKKRRQRRRGQRNPNKKWS. | (SEQ ID NO:3) |

NHS-Fluorescein was purchased from Pierce Chemical Co. (Rockford Ill.). To label the peptides with the fluorescent dye, 500 μg of peptide was mixed with 250 μg of NHS-Fluorescein in DMSO, incubated for 2 hours on ice, purified by a PD10 column and eluted in 0.75 ml fractions of PBS buffer. The labeling and purification of the peptide was examined by HPLC. Fractions 2 and 3 had the majority of the labeled peptides.

293T (human embryonic kidney) cells were grown in Dulbecco's modified Eagle's medium (Life Technologies, Rockville, Md.) supplemented with 10% FBS, 100 units/ml penicillin, and 100 μg/ml of streptomycin in a 37° C. CO₂ incubator. Cells were regularly passed to maintain growth. Twenty-four hours before transfection, cells were trypsinized and plated on 96-well plates (100 μl/well) in the above medium without the antibiotics. Ten μl of fraction 2 was added to 293T cells at 15% confluency in serum-containing medium. Fluorescence microscopy was performed at 12 hours, after changing medium and washing cells.

Fluorescence was observed throughout the transducted cells. Examination of a single cell revealed that the cell divided by 24 hours, and that the resulting two daughter cells also showed evenly distributed fluorescent light, but of lower intensity due to dilution. This experiment demonstrates that the peptide encompassing Nuc and a known signal peptide effectively cross the cell membrane of eukaryotic cells.

### Example 2

### Transfection Abilities of Peptide Tan

TAN (SEQ ID NO:3) at 0.1 to 5 μg per 96-well transfection was mixed with 0.1 μg plasmid pEGFP-N1 (BD ClonTech) for 5 min, diluted in 40 μl DMEM, then added to cells. Fluorescence microscopy was performed at different time points to observe the expression of GFP as the indication of transfection event. Note that by using GFP as reporter, the total transfection efficiency was underestimated due to certain percentage of cells would not express the GFP to levels high enough for detection. A set of photographs demonstrated the dose effect of peptide TAN in combination with Ca⁺⁺ on transfection, indicating that there is a specific effect by TAN under a given Ca⁺⁺ concentration and vise versa.

### Example 3

### Transfection Abilities of Peptide Tan in Combination with Calcium Ion

Different concentrations of TAN (SEQ ID NO:3) and CaCl₂ were tested in combination for transfection. TAT (SEQ ID NO:1) and another peptide, HT 31 (a widely used PKA-anchor interaction interruption peptide) were included as controls. Generally, experiments were performed similarly as in the above examples except that peptide was added to DNA first, followed by addition of CaCl_{2.} Nonetheless, an order-of-addition experiment indicated that adding peptide, CaCl_{2,} or DNA in any order result in similar transfection efficiency. As control, TAT together with CaCl₂ caused cells to lyse, while the HT31 showed no effect.

As little as 0.1 μg of peptide in the presence of 15 mM of calcium resulted in high transfection efficiency. TAN was not examined at amounts less than 0.1 μg, but may nevertheless mediate transfection to similar or higher levels. The “charge ratio”, defined as the ratio between the total number of the positive charges on the peptide and the total negative charges on the DNA, was close to 1:1. This ratio is lower than typical cationic peptide-mediated transfections, agreeing with the notion that the binding of this peptide is not mainly through electrostatic interaction. The amount of DNA used to achieve the demonstrated levels of transfection using our peptide transfection system is also lower than those normally suggested for commercial transfection reagents.

The effects of calcium ion was dose-dependent, with a bell curve peaking at about 30 mM calcium with 0.5 μg TAN peptide. Calcium phosphate precipitation is one of the oldest methods of transfection. It is possible that the effect of Ca⁺⁺ on transfection in the TAN system was due to the interaction between Ca⁺⁺ and phosphate presented by the medium. To test this, transfection experiments were performed with TAN in PBS or Mono-Q™ column desalted H₂O. The inclusion of phosphate in the reaction actually decreased transfection efficiency, thus demonstrating that the effect of combining TAN and calcium ion was not enhanced by the presence of phosphate, and indicating that TAN likely acts by a mechanism different from calcium phosphate precipitation.

This result indicates that calcium in its free ionic state, instead of as a phosphate precipitate, is the functional component in enhancing the TAN-mediated transfection. In addition, with different amounts of the peptide, the optimum concentration of calcium ion can change, suggesting a defined relationship between the two factors in forming cell transfecting DNA complexes. Furthermore, in forming DNA precipitates by a typical calcium phosphate transfection protocol, the concentration of calcium is 0.122 M, whereas in the reactions exemplified herein, the concentration was about 10-fold lower. Higher calcium ion concentrations sometimes resulted in severe cell toxicity.

MgCl₂, NaCl and other salts were included as control and showed no effect on enhancing TAN-mediated transfection, indicating specificity of calcium ion. A time course was performed in order to find out the optimum incubation time period of forming TAN-DNA-Ca⁺⁺ complexes. The reaction appeared to reach the highest transfection effect quickly (within 1 min, and peaking by 5 min), which is advantageous for transfection, suggesting a preferable kinetics for most applications in comparison with other methods.

### Example 4

### Transfection Enhancing Effects of Peptide Tan in Combination with other Transfection Reagents

To determine whether TAN increased transfection efficiency when used as an additive to other transfection systems, several commercial transfection reagents were used accordingly to the manufacturers' protocols, but with TAN (SEQ ID NO:3) or control peptides added to the DNA first. Preliminary data indicated that TAN increased transfection efficiency of various transfection reagents under different conditions. Five μg of TAN (SEQ ID NO:3) or TAT (SEQ ID NO:2) was added to DNA before performing transfection with TransIt-Oligo™ reagent (Mirus; Madison Wis.). After incubating peptide with DNA for 5 min, 0.5 μl of TransIt-Oligo™ reagent diluted in 10 μl of DMEM was added and the mixture was further incubated for 15 min before overlaying onto 293T cells as described above. Several fold higher GFP expression was consistently observed with TAN than with control peptides. These results demonstrate that TAN (SEQ ID NO:3) can increase the transfection ability of other transfection systems, for example, by accompanying DNA into cells and nuclei.

### Example 5

### Transfection of Difficult-To-Transfect Cells Using the Tan Transfection System

In order to test whether transfection by the novel peptide could be applied to other cell types, including cells that are known to be difficult to transfect with existing methods, several different cell lines were examined using the procedures described above. GFP expression was enhanced by TAN (SEQ ID NO:3) in a TransIt-Oligo™ reagent-mediated transfection. In this case, although the total number of GFP-positive cells did not appear to increase in the presence of TAN, the cells that were transfected using TAN were much brighter as compared to those transfected without TAN. The experiments were done in 24-well plate with 3T3-L1 cells 15 days post-induction, a time point when transfection is normally extremely difficult. Note that the amount of DNA used was also very limited (0.2 μg in reaction 1-3, 0.5 μg in reaction 4).

Transfection was examined with monkey kidney cell Vero-E6 (ATCC) cell line, which is routinely used for human disease related viral infection, e.g. SARS, as a drug testing model system. Vero-E6 cells with limited passages from original ATCC stock culture were grown in 96-well plate at about 30% confluency. Transfections were performed with 0.1 μg of plasmid pEFGP-N1 as described above. Two doses of TransIt-Oligo™ reagent were tested, but neither provided good transfection under this conditions. However, with increasing amount of TAN (SEQ ID NO:3), the transfected cells were more prominent. In parallel control experiments, TAT (SEQ ID NO:2) did not show such enhancement.

### Example 6

### Tan Compacts Plasmid DNA and Calcium Facilitates Complex Formation

In order to examine whether TAN could directly interact with the transfected plasmid DNA as predicted by the transfection results, band-shift assays were performed. Different amount of TAN (SEQ ID NO:3) dissolved in H₂O was mixed with 0.5 μg of plasmid with or without 15 mM CaCl₂, incubated for 5 min, followed by addition of 2 μl of DNA loading dye (30% glycerol), and loaded to 0.7% agarose gel. The EB-containing gel was run for about 45 min at 120 volts before the picture taken under UV lights.

Two types of changes were observed as result of TAN: 1) some plasmid stayed in the wells, as seen with almost all published reports on such peptide band-shift assays, presumably due to the aggregation of DNA induced by peptide binding; and 2) a certain amount of plasmid migrated more quickly, as a distinct band, than even the supercoiled plasmid, suggesting they were compacted but not in any large aggregates. Significantly, in the presence of calcium ion, the complex became more intense, while all other bands decreased. These results indicate that calcium ion can stabilize the TAN-DNA complex, thus reducing aggregation. Taken together with the transfection data, these results indicate that calcium ion can affect TAN-mediated transfection, at least in part, at the step of complex formation.

### Example 7

### Tan Binds Linear Cassettes of Sirna-expressing DSDNA

The ability of TAN (SEQ ID NO:3) to bind short linear dsDNA also was examined. The DNA tested was an approximately 300 bp PCR product that is used to express small interfering RNAs (siRNAs). The experiments were done similarly to those described in Example 6, except that the reactions were allowed to proceed to 30 min and the gel was 2%. TAN (SEQ ID NO:3) was found to bind to the linear dsDNA in a dose-dependent manner, with a Kd of approximately 1.5×10⁻⁴M⁻¹ which is in the same range as was observed for the double stranded plasmid DNA. Interestingly, the complexes formed between DNA and TAN migrated more slowly than the free DNA, which is different from those between plasmid and TAN. This difference may be due to a difference in the relative amount of DNA in each type of complex or to the structural constrains of linear versus circular DNA. As controls, peptide HT31 did not bind the DNA at similar concentrations. TAT (SEQ ID NO:2) also did not bind at the lower concentrations, and quickly precipitated DNA as concentration increased slightly. The TAN peptide also enhanced the RNAi effects when used in transfection aimed at causing gene silencing by the siRNA-expressing cassettes. Because these cassettes express siRNAs under the control of Pol III promoter as apposed to Pol II in GFP expression unit, it is therefore likely that peptide assisted transfection (PAT) is independent of promoter used, as expected.

References Cited:

Each of the following references is incorporated herein by reference:
Aronsohn, A. I., and Hughes, J. A. (1998). Nuclear localization signal peptides enhance cationic liposome-mediated gene therapy, J Drug Target 5, 163-9.
Becker-Hapak, M., McAllister, S. S., and Dowdy, S. F. (2001). TAT-mediated protein transduction into mammalian cells, Methods 24, 247-56.
Belting, M., and Petersson, P. (1999). Intracellular accumulation of secreted proteoglycans inhibits cationic lipid-mediated gene transfer. Co-transfer of glycosaminoglycans to the nucleus, J Biol Chem 274, 19375-82.
Brunner, S., Sauer, T., Carotta, S., Cotten, M., Saltik, M., and Wagner, E. (2000). Cell cycle dependence of gene transfer by lipoplex, polyplex and recombinant adenovirus, Gene Ther 7, 401-7.
Coeytaux, E., Coulaud, D., Le Cam, E., Danos, O., and Kichler, A. (2003). The cationic amphipathic alpha-helix of HIV-1 viral protein R (Vpr) binds to nucleic acids, permeabilizes membranes, and efficiently transfects cells, J Biol Chem 278, 18110-6.
Debs, R. J., Freedman, L. P., Edmunds, S., Gaensler, K. L., Duzgunes, N., and Yamamoto, K. R. (1990). Regulation of gene expression in vivo by liposome-mediated delivery of a purified transcription factor, J Biol Chem 265, 10189-92.
Dheur, S., Dias, N., van Aerschot, A., Herdewijn, P., Bettinger, T., Remy, J. S., Helene, C., and Saison-Behmoaras, E. T. (1999). Polyethylenimine but not cationic lipid improves antisense activity of 3′-capped phosphodiester oligonucleotides, Antisense Nucleic Acid Drug Dev 9, 515-25.
Dheur, S., and Saison-Behmoaras, T. E. (2000). Polyethyleneimine-mediated transfection to improve antisense activity of 3′-capped phosphodiester oligonucleotides, Methods Enzymol 313, 56-73.
Escriou, V., Carriere, M., Bussone, F., Wils, P., and Scherman, D. (2001). Critical assessment of the nuclear import of plasmid during cationic lipid-mediated gene transfer, J Gene Med 3, 179-87.
Felgner, P. L., Tsai, Y. J., Sukhu, L., Wheeler, C. J., Manthorpe, M., Marshall, J., and Cheng, S. H. (1995). Improved cationic lipid formulations for in vivo gene therapy, Ann N Y Acad Sci 772, 126-39.
Ferrari, S., Pettenazzo, A., Garbati, N., Zacchello, F., Behr, J. P., and Scarpa, M. (1999). Polyethylenimine shows properties of interest for cystic fibrosis gene therapy, Biochim Biophys Acta 1447, 219-25.
Fritz, J. D., Herweijer, H., Zhang, G., and Wolff, J. A. (1996). Gene transfer into mammalian cells using histone-condensed plasmid DNA, Hum Gene Ther 7, 1395-404.
Godbey, W. T., and Mikos, A. G. (2001). Recent progress in gene delivery using non-viral transfer complexes, J Control Release 72, 115-25.
Godbey, W. T., Wu, K. K., and Mikos, A. G. (2001). Poly(ethylenimine)-mediated gene delivery affects endothelial cell function and viability, Biomaterials 22, 471-80.
Gonzalez, H., Hwang, S. J., and Davis, M. E. (1999). New class of polymers for the delivery of macromolecular therapeutics, Bioconjug Chem 10, 1068-74.
Grove, A., and Lim, L. (2001). High-affinity DNA binding of HU protein from the hyperthermophile Thermotoga maritima, J Mol Biol 311, 491-502.
Haines, A. M., Irvine, A. S., Mountain, A., Charlesworth, J., Farrow, N. A., Husain, R. D., Hyde, H., Ketteringham, H., McDermott, R. H., Mulcahy, A. F., et al. (2001). CL22 - a novel cationic peptide for efficient transfection of mammalian cells, Gene Ther 8, 99-110.
Kamata, H., Yagisawa, H., Takahashi, S., and Hirata, H. (1994). Amphiphilic peptides enhance the efficiency of liposome-mediated DNA transfection, Nucleic Acids Res 22, 536-7.
Kim, H. H., Lee, W. S., Yang, J. M., and Shin, S. (2003). Basic peptide system for efficient delivery of foreign genes, Biochim Biophys Acta 1640, 129-36.
Lamb, B. T., and Gearhart, J. D. (1995). YAC transgenics and the study of genetics and human disease, Curr Opin Genet Dev 5, 342-8.
Lee, H., Kim, T. H., and Park, T. G. (2002). A receptor-mediated gene delivery system using streptavidin and biotin-derivatized, pegylated epidermal growth factor, J Control Release 83, 109-19.
Liu, Y., Liggitt, D., Zhong, W., Tu, G., Gaensler, K., and Debs, R. (1995). Cationic liposome-mediated intravenous gene delivery, J Biol Chem 270, 24864-70.
Liu, Y., Mounkes, L. C., Liggitt, H. D., Brown, C. S., Solodin, I., Heath, T. D., and Debs, R. J. (1997). Factors influencing the efficiency of cationic liposome-mediated intravenous gene delivery, Nat Biotechnol 15, 167-73.
Malone, R. W., Felgner, P. L., and Verma, I. M. (1989). Cationic liposome-mediated RNA transfection, Proc Natl Acad Sci USA 86, 6077-81.
Marschall, P., Malik, N., and Larin, Z. (1999). Transfer of YACs up to 2.3 Mb intact into human cells with polyethylenimine, Gene Ther 6, 1634-7. Mo, Y. Y., Wang, C., and Beck, W. T. (2000). A novel nuclear localization signal in human DNA topoisomerase I, J Biol Chem 275, 41107-13.
Remy, J. S., Kichier, A., Mordvinov, V., Schuber, F., and Behr, J. P. (1995). Targeted gene transfer into hepatoma cells with lipopolyamine-condensed DNA particles presenting galactose ligands: a stage toward artificial viruses, Proc Natl Acad Sci USA 92, 1744-8.
Reya, T., Duncan, A. W., Ailles, L., Domen, J., Scherer, D. C., Willert, K., Hintz, L., Nusse, R., and Weissman, I. L. (2003). A role for Wnt signalling in self-renewal of haematopoietic stem cells, Nature 423, 409-14.
Richardson, S. C., Kolbe, H. V., and Duncan, R. (1999). Potential of low molecular mass chitosan as a DNA delivery system: biocompatibility, body distribution and ability to complex and protect DNA, Int J Pharm 178, 231-43.
Rittner, K., Benavente, A., Bompard-Sorlet, A., Heitz, F., Divita, G., Brasseur, R., and Jacobs, E. (2002). New basic membrane-destabilizing peptides for plasmid-based gene delivery in vitro and in vivo, Mol Ther 5, 104-14.
Saito, K., Kikuchi, T., and Yoshida, M. (1999). The mechanism of sequence non-specific DNA binding of HMG1/2-box B in HMG1 with DNA, Protein Eng 12, 235-42.
Sandgren, S., Cheng, F., and Belting, M. (2002). Nuclear targeting of macromolecular polyanions by an HIV-Tat derived peptide. Role for cell-surface proteoglycans, J Biol Chem 277, 38877-83.
Schwarze, S. R., Ho, A., Vocero-Akbani, A., and Dowdy, S. F. (1999). In vivo protein transduction: delivery of a biologically active protein into the mouse, Science 285, 1569-72.
Scott, E. S., Wiseman, J. W., Evans, M. J., and Colledge, W. H. (2001). Enhanced gene delivery to human airway epithelial cells using an integrin-targeting lipoplex, J Gene Med 3, 125-34.
Shoeman, R. L., Hartig, R., and Traub, P. (1999). Characterization of the nucleic acid binding region of the intermediate filament protein vimentin by fluorescence polarization, Biochemistry 38, 16802-9.
Tang, F., and Hughes, J. A. (1999). Synthesis of a single-tailed cationic lipid and investigation of its transfection, J Control Release 62, 345-58.
Toth, J., Boszormenyi, I., Majer, Z. S., Laczko, I., Malvy, C., Hollosi, M., and Bertrand, J. R. (2002). A two step model aimed at delivering antisense oligonucleotides in targeted cells, Biochem Biophys Res Commun 293, 18-22.
Watnick, R. S., Herring, S. C., Palmer, A. G., 3rd, and Gottesman, M. E. (2000). The carboxyl terminus of phage HK022 Nun includes a novel zinc-binding motif and a tryptophan required for transcription termination, Genes Dev 14, 731-9.
Wickham, T. J., Carrion, M. E., and Kovesdi, I. (1995). Targeting of adenovirus penton base to new receptors through replacement of its RGD motif with other receptor-specific peptide motifs, Gene Ther 2, 750-6.
Wyman, T. B., Nicol, F., Zelphati, O., Scaria, P. V., Plank, C., and Szoka, F. C., Jr. (1997). Design, synthesis, and characterization of a cationic peptide that binds to nucleic acids and permeabilizes bilayers, Biochemistry 36, 3008-17.
Yoshimura, K., Rosenfeld, M. A., Seth, P., and Crystal, R. G. (1993). Adenovirus-mediated augmentation of cell transfection with unmodified plasmid vectors, J Biol Chem 268, 2300-3.

Although the invention has been described with reference to the above example, it will be understood that modifications and variations are encompassed within the spirit and scope of the invention. Accordingly, the invention is limited only by the following claims.

1. A peptide having an amino acid sequence consisting of QRNPNKKWS (SEQ ID NO:1).

2. The peptide of claim 1, which comprises a peptide fragment of a Nun polypeptide.

3. A fusion protein, comprising the peptide of SEQ ID NO:1 operatively linked to a heterologous peptide.

4. The fusion protein of claim 3, wherein the heterologous peptide comprises a cellular localization domain.

5. The fusion protein of claim 4, wherein the cellular localization domain comprises a nuclear localization signal.

6. The fusion protein of claim 4, wherein the nuclear localization signal comprises SEQ ID NO:4 or SEQ ID NO:8.

7. The fusion protein of claim 4, wherein the cellular localization signal comprises a human immunodeficiency virus (HIV) TAT peptide or an HIV gp41 peptide.

8. The fusion protein of claim 7, wherein the HIV TAT peptide comprises an amino acid sequence as set forth in SEQ ID NO:2, and wherein the HIV gp41 peptide comprises an amino acid sequence as set forth in SEQ ID NO:5.

9. The fusion protein of claim 7, further comprising a nuclear localization signal.

10. The fusion protein of claim 3, which has an amino acid sequence as set forth in SEQ ID NO:3.

11. The fusion protein of claim 3, which further comprises an endoprotease recognition site, an endosomolytic peptide, or a combination thereof.

12. A composition, comprising the peptide of claim 1 and a heterologous polypeptide.

13. A composition, comprising the peptide of claim 1 and a nucleic acid molecule.

14. The composition of claim 13, wherein the nucleic acid molecule comprises a double stranded nucleic acid molecule.

15. The composition of claim 13, wherein the nucleic acid molecule comprises DNA or RNA.

16. A composition, comprising the peptide of claim 1 and divalent calcium ions.

17. The composition of claim 13, further comprising divalent calcium ions.

18. A kit, comprising the peptide of claim 1.

19. The kit of claim 18, wherein the peptide of claim 1 is operatively associated with a heterologous polypeptide.

20. The kit of claim 19, wherein the peptide of claim 1 and the heterologous polypeptide comprise a fusion protein.

21. The kit of claim 20, wherein the fusion protein comprises, in operative linkage, the peptide of claim 1 operatively linked to a peptide having an amino acid sequence comprising SEQ ID NO:2 or SEQ ID NO:5.

22. The kit of claim 21, wherein the fusion protein has an amino acid sequence as set forth in SEQ ID NO:3.

23. The kit of claim 18, further comprising at least one heterologous polypeptide, which can be operatively linked to or operatively associated with the peptide of claim 1.

24. The kit of claim 23, which comprises a plurality of heterologous polypeptides, wherein at least two heterologous polypeptides of the plurality are different.

25. The kit of claim 18, further comprising a transfection reagent.

26. The kit of claim 25, wherein the transfection reagent comprises divalent calcium ions.

27. A solid substrate, which comprises at least one peptide of claim 1.

28. The solid substrate of claim 27, which comprises a plurality of peptides of claim 1.

29. The solid substrate of claim 28, wherein the peptides of the plurality are in array.

30. The solid substrate of claim 28, wherein the peptide of claim 1 comprises a complex with a nucleic acid molecule.

31. A method of transfecting a cell, comprising contacting the cell with the peptide of claim 1 and a nucleic acid molecule under conditions sufficient from cell transfection.

32. The method of claim 31, wherein said conditions comprise further contacting the cell with divalent calcium ions.

33. The method of claim 31, wherein the peptide of claim 1 further comprises an operatively associated heterologous polypeptide.

34. The method of claim 31, wherein the peptide of claim 1 is attached to a solid substrate.

35. A method of transfecting a cell, comprising contacting the cell with the fusion protein of claim 3 and a nucleic acid molecule under conditions sufficient from cell transfection.

36. The method of claim 35, wherein the heterologous polypeptide comprises an HIV TAT peptide or an HIV gp41 peptide.

37. The method of claim 36, wherein the fusion protein further comprises a nuclear localization signal.

38. The method of claim 35, wherein the fusion protein has an amino acid sequence as set forth in SEQ ID NO:3.

39. The method of claim 35, wherein said conditions comprise further contacting the cell with divalent calcium ions.


SEQUENCE LISTING (8 sequences)

SEQ ID NO: 1 - protein, 9 aa, HK 022 Phage
QRNPNKKWS

SEQ ID NO: 2 - protein, 11 aa, Human immunodeficiency virus
YGRKKRRQRRR

SEQ ID NO: 3 - protein, 21 aa, Artificial sequence
  Fusion peptide
YGRKKRRQRRRGQRNPNKKWS

SEQ ID NO: 4 - protein, 8 aa, Artificial sequence
  Nuclear localization signal
PKKIKTED

SEQ ID NO: 5 - protein, 17 aa, Human immunodeficiency virus
GALFGGFLGAAGSTMGA

SEQ ID NO: 6 - protein, 6 aa, Artificial sequence
  Cathepsin D recognition sequence
GGFLGF

SEQ ID NO: 7 - protein, 23 aa, Influenza virus
GLFEAIEGFIENGWEGMIDGWYG

SEQ ID NO: 8 - protein, 7 aa, Artificial sequence
  Nuclear localization signal
KKIKTED